(19) United States
(12) Patent Application Publication
(10) Pub. No.: US 2007/0192137 A1
(43) Pub. Date: Aug. 16, 2007

(54) ACCESS CONTROL IN AN ELECTRONIC MEDICAL RECORD SYSTEM

(76) Inventor: Mark P. Ombrellaro, Bellevue, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/670,401

(22) Filed: Feb. 1, 2007

Related U.S. Application Data
(60) Provisional application No. 60/763,976, filed on Feb. 1, 2006.

(51) Int. Cl. G06Q 10/00; A61B 5/00

(52) U.S. Cl. 705/2; 600/300

(57) ABSTRACT

A patient-centric medical record access and control method is disclosed wherein the patient has full read privileges and limited write privileges to the patient's electronic medical record account, and further wherein the patient can designate specific individual and institutional healthcare providers with access to the patients EMR account, and can limit access by previously designated providers. In an embodiment of the method, the patient is provided with a selectable list of providers, and selects one or more providers for access. The selected providers receive access, notification and demographic information on the patient. In another aspect, the patient can removably designate a guardian to have access generally the same as the patient's access to the EMR account. In another aspect of the invention, the selected providers can optionally terminate their write access to the EMR account, and termination notification is provided to the patient.

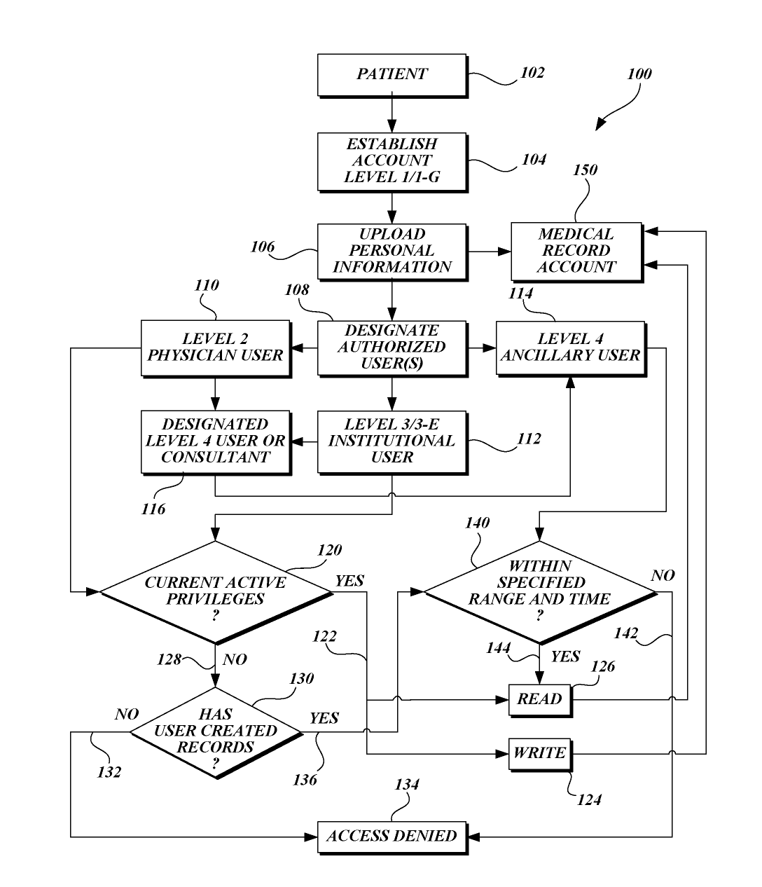

### CROSS-REFERENCE TO RELATED APPLICATION

This application claims the benefit of U.S. Provisional Patent Application No. 60/763,976, filed Feb. 1, 2006, the disclosure of which is hereby expressly incorporated by reference in its entirety, and priority from the filing date of which is hereby claimed under 35 U.S.C. § 119.

### BACKGROUND

Creation of a unified electronic medical record system, (or electronic health record system), is a focus of worldwide attention in medical informatics. With an electronic medical record system, the speed and efficiency of medical data acquisition and preservation as well as the accuracy and availability of critical health-related documents can be improved. Clearly, improving the accuracy and availability of health and medical information will lead to improvements in patient safety and the overall quality of health care. While unlimited, rapid access to this type of information provides clear advantages, the widespread acceptance of widely-available data access has been hindered by the sensitive and personal nature of health information. The distribution of medical record information is protected by the Health Insurance Portability and Accountability Act of 1996, sometimes referred to as HIPAA, which addresses the integrity, confidentiality and availability of electronic health information as it is collected, stored, and transmitted.

Currently, the goal of health and medical record information reform is to move to an entirely paperless digital system. Various electronic medical record software systems have been developed, but no single system is available that will serve the needs of all practitioners. A low cost widely available solution will be required if a truly universal, centralized medical information clearinghouse is to evolve. Ideally, a universal medical record system would utilize a common internet-based platform that will allow the greatest potential for access among all potential users. An internet-based system that fosters ease of access to sensitive personal information requires a system of controls that will allow only authorized individuals to access the information while preventing all other individuals from having access to it. Described below is a novel patient-based system architecture that allows health care providers and other appropriate individuals or organizations the ability to record and view a patient's personal medical information while preventing all others from accessing that information.

As contemplated by the present invention, medical record documents are unlike other types of electronic documents in that they are not “single owner” documents. Rather, medical records are considered to be co-owned by both the patient who is the subject of the medical record document, and the health care provider generating and having custody of the medical record document. This dual ownership of a digital medical record document adds significant complexity with respect to ownership, control, access, and security of the document itself. Resolution of these issues is particularly important for medical record documents due to the dual goals of providing healthcare workers with ready access to the documents, and protecting the sensitive information from others by limiting access to individuals without authorization.

The present system begins with the need to exchange medical information between two or more people. For the purposes of illustration the present system is described with reference to what is currently believed to be the most useful application, wherein the donor or subject of the information will typically be referred to as the “patient” and a typical recipient of the information will typically be referred to as the “physician” or “health care provider.” When referring generically to either a patient or physician herein, we typically will refer to a “user.” However, it is not intended that the present invention would be limited strictly to patient-physician information exchanges.

To participate in the information exchange process, either as “patient,” “physician” or any other type of healthcare provider or representatives of the patient or physician, the user will require a computer, access to the Internet or other suitable electronic network, a software application with the functionality described herein, and appropriate peripheral devices to take full advantage of the system's complete functionality.

In healthcare informatics and patient information systems, there are generally two different types of “owners” of health information: the patient and the health care provider. In a preferred model, the patient “owns” their personal health information, such as the information content of the patient's medical records. The medical records may include any document or image that conveys health-related information about the individual, and may be, for example, a paper-based, film, or digital document. In the preferred model, the generating healthcare provider, for example the physician, owns the actual healthcare documents that the physician creates, irrespective of the content. With this model of a dual ownership system, the patient is allowed unrestricted access to read and review their medical information (content), including the right to obtain a copy of their medical information from the health care provider(s). The healthcare provider controls the actual medical document itself.

This dual-ownership model, in combination with the move to digital or electronic medical record systems presents many key issues relating to the control and integrity of the healthcare medical records, as well as thorny issues relating to both granting and restricting access to the medical record information. For example, although the patient is the owner of the information in the medical record, it is clearly not desirable for the information entered by a professional healthcare provider be modifiable by the patient, or by others having access to the medical records. However, a particular patient may elect to change healthcare providers a number of times over a period of time, and may wish to change a particular healthcare provider's access to the patient's medical records. In addition a patient may wish to grant limited access to non-medical third parties, such as legal guardians, insurers or legal professionals under certain circumstances. As will be appreciated by persons of skill in the art, questions and issues relating to levels of access (e.g., read only, read/write, etc.), temporal and subject matter limitations to access for various users, and the like, are many and significant.

The first critical issue, of course, is who has a right to access the health care information, and including access as a function of time.

From the patient's perspective, key access issues include:
Who, among the universe of health care providers, is allowed access to view their medical information, and what is the time frame for that access? These will be referred to as practitioner read only privileges.
Which health care providers have the ability to add to or input data into the medical record, and what is the time frame for which this access is allowed? This is referred to herein as practitioner read/write privileges.
The designation of ancillary health care providers (health care staff, insurers or attorneys, for example) that may view their records in a read-only, time-dependent format (ancillary read only privileges).
The patient's own ability to update their personal information as necessary, and mechanisms for keeping an archive of previous entries so a timeline of all entries can be identified (patient read/limited write privileges).
The patient's own ability to view the entirety of their medical records in the universal system (patient read only privileges).
The ability for a patient to designate a “guardian” or authorized agent (such as a family member) to view their health and medical record information on their behalf and/or in case of emergency.
   From the healthcare provider's perspective, key access issues include
Who's health care records, among the universe of patients, is the practitioner allowed active access to view their medical information, and during what time frame is that access allowed. (practitioner read only privileges.)
Which of the patient's records is the practitioner allowed to add to or otherwise input data into, and a time frame for which such access is allowed (practitioner read/write privileges)?
What constraints exist on the ability or the healthcare provider to share a patient's medical information with other providers for consultation, and with ancillary health care providers (insurers or attorneys, for example) in a read-only and time-dependent format (ancillary read only privileges).
The ability to access any patient's health information in case of emergency.
The ability to view the entirety of the medical records that they developed across all patients and all time frames.

A method for providing and controlling access to medical records, and medical record information, in an electronic medical record system, is disclosed herein that provides a solution to many of the issues discussed above.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

The FIGURE is a flow diagram outlining a particular embodiment of the invention disclosed herein.

### DETAILED DESCRIPTION

The medical records access and management methodology described herein is initially conceived for use in a web-based electronic medical record system. A particularly advantageous electronic medical record system is the multifunction telemedicine system disclosed in co-pending U.S. patent application Ser. No. 11/061,490 and published in U.S. Pat. Publ. No. 20050149364A1, the disclosure of which is hereby incorporated by reference in its entirety. Although it is not required for the present invention, a preferred electronic medical record system would include integrated on-line patient-provider communications tools that allow for the exchange of medical information between health care stakeholders in a universally accessible, secure, and efficient fashion.

Considering the issues discussed in the background section above, the hierarchy of information access is divided into four basic levels of control.

Level 1 Access: Account Owner Access

Level 1 access to the medical record account is given to each individual patient, as they are the “owner” of their personal information. It is anticipated that there is only one true “owner” on each account. Level 1 access allows full, unrestricted “read” access to all personal information within that account and cannot be disabled. Account owner Level 1 access provides full “read” privileges for each medical record and other electronic document within the patient's account at any time. The account owner Level 1 access also includes limited “write” privileges. For example, with Level 1 access the patient can update demographic information in the patient's health care record, personal health history (e.g., past medical history, such as hospitalizations/surgeries), medication list, allergies, social history, family history, habits, review of systems checklist, and other data that a healthcare provider customarily obtains directly from the patient.

In a preferred embodiment of the method, any saved change in the record made by the account owner generates a permanent entry into the account, and any prior data amended or deleted by the saved change is archived. Changes to the medical record account made by the account owner are date, time, and author stamped, and the prior records are archived and retrievable.

Level 1-G Access: Account Guardian Access

On medical record accounts for persons under the age of eighteen, and for patients who cannot act for themselves and have legal guardians appointed, the parent(s) and/or legal guardian are given Level 1-G access. Level 1-G access is essentially the same as the Level 1 access, and allows an authorized parent or guardian to act on behalf of the Level 1 access, account owner. Level 1-G access allows full read access to the information in the medical record account, but can be disabled by the account owner with Level 1 access. Level 1-G access has full “read” privileges for each document or bit of information within the account at any time. The Level 1-G guardian also has the limited “write” privileges described above for the Level 1 account owner. Any saved change, creates a permanent entry into the account. Changes made to the account by the Level 1-G guardian are date, time, and author stamped, and generate a new record, with retrievably archiving similar to the account owner changes, as discussed above.

Level 2 Access: Author Access

Level 2 access is given to the authoring of the medical records, for example the physician (or other healthcare provider), who is considered to be the “owner” of the documents that they create. A physician with Level 2 access may read all documents within the medical record as well as create new medical record documents to add to the medical record account. Physicians with Level 2 access may update information or add data to any aspect of the medical record account in an unrestricted fashion. When any change to the medical record is made and saved, a permanent entry is created into the medical record that is time, date, and author stamped, and any modified or deleted data is archived in a retrievable manner. Level 2 access can be turned on or off at any time by a person with Level 1 or Level IG access.

Level 3: Institutional Access

Level 3 access may be provided in the institutional or group practice setting, wherein patients will typically receive a variety of healthcare services that may come, for example, from any of the spectrum of inpatient, emergency, laboratory, imaging, and some types of outpatient services. Currently, a patient gives permission to be treated by the institutional providers under a blanket of the institutional permission rather than on a provider-by-provider basis. Medical records generated in conjunction with these services are maintained at the institution and are not typically kept separately by the individual physician or other healthcare provider. A typical institutional access provider, for example, would be hospital-based service providers such as professionals in anesthesia, pathology, and radiology. Under institutional Level 3 access, all providers who are registered as care providers under an institutional license are granted access to the patient's healthcare record. In the rare circumstance wherein a specific provider may be given access to records via an institutional license (Level 3 access) but has previously had their access “turned off” by the patient in the non-institutional setting (i.e., Level 2 access), that provider's access would remain restricted.

Level 3 access allows healthcare providers who are authorized under an institutional account to read all documents within the medical record as well as to create medical documents or add to the medical record as an agent of the institution. This can be done in an unrestricted fashion. When any change to the medical record is made and saved, a permanent entry is created into the medical record that is time, date, and author, and location stamped. Documents generated under an institutional license are generally the property of the institution. In the preferred embodiment, for physicians with both Level 2 access and Level 3 access, documents will be tagged to both accounts or owners and the institution and the particular physician is considered to be a “co-owners” of the medical document. Level 3 access can be turned on or off at any time by a person with Level 1 or Level 1-G access.

Level 3-E Access: Emergency Access

It is anticipated that a patient may present to an institution under emergency circumstance and may be unconscious or otherwise non-responsive. In such an emergency, where the patient is in need of health care from an institution or physician who has no prior account access authorization, an emergency access protocol is established, referred to herein as Level 3-E access. Patients who wish to enable the emergency access protocol must first turn this account function on and establish a secondary account login and password sequence. In a preferred embodiment, when turning this account function on a patient would enter a biometric identifier that could be used to activate Level 3-E access, for example the system might require the patent upload a fingerprint scan of the right index finger into their account. The right index finger is chosen for example purposes but any finger or other type of biometric log-on process is a viable option. Once the emergency biometric log-on function is activated, if a patient shows up unconscious, in an emergency room for example, the right index finger can be physically scanned to establish a biometric, and an emergency registration menu will appear. Emergency institutional Level 3-E access can be obtained allowing the accessing healthcare providers to view the patient's medical information.

In a preferred embodiment of the present method, institutional medical providers may independently register for an institutional account to a central repository of medical records. If emergency access is obtained in an institution without an institutional account, the medical record documents are in “read only” mode where providers have access to the patient's current medical information. If the institution has an established institutional account, then account access includes the ability to both read and write to the electronic medical record account.

Level 4 Access: Ancillary Services Access

It is also contemplated that during the normal course of business it may sometimes be desirable to provide access to specific documents within a medical record, for example for purposes of medical consultation, or at the legitimate request of authorized insurers, legal representatives, or other third parties. For such ancillary services access, the present method contemplates a more limited access level, referred to as Level 4 access. Rather than direct access to the entirety of the medical record, Level 4 access allows indirect access to the required medical information, preferably via a document reader.

In the current embodiment, users with Level 1, Level 2, or Level 3 access can designate the appropriate ancillary user, assign that user Level 4 access, and then select the document or group of documents to send to a suitable document reader. Suitable readers are well-known in the art, and/or may be readily developed by persons of ordinary skill in the art. Level 4 access privileges are user-specific, document-specific, and time-limited. Each designation of Level 4 access represents a unique document transaction. Therefore, for an ancillary user to view additional documents not in the original designation, a new document range needs to be assigned by reselecting the user, assigning the reselected user access to the additional documents, and identifying the specific additional documents to be viewed. Level 4 document access is strictly “read only” and none of the records can be changed by a Level 4 user. Level 4 access can be turned off by the Level 1, or Level 1-G user, or by the user who granted that particular Level 4 access. Level 4 users are not able to assign Level 4 access to others, forward documents to others, or access other aspects of the medical record account.

It is contemplated that a modified Level 4 status may also be employed for non-physician users of institutional or group practice accounts. For example, personnel such as administrative, scheduling, or other front or back office employees may need access to patient information, yet be limited in their ability to add information directly into the health record account.

As discussed below, a software application utilizing the proposed access authorization methodology will typically include two components: a physician-side application component and a patient-side application component. Each of the components may be, for example, a freestanding software application or may be imbedded within a web page or web based document. Users of either component log on to a secure server system and must be authenticated, for example by one or more of several methods including a secure user name and password and/or biometric identification system. Once a user is logged into the component and the user has been authenticated, the various aspects of the software's functionality may be utilized. Described below is a currently preferred implementation of the access method for the electronic medical record system contemplated by the present invention. It will be appreciated that the general concepts may be applied to any number of situations, for example banking, finance or securities transactions; medical information, or legal industry; where personal information may need to be disseminated to a second or third party.

### Software Functionality

In the present embodiment, the software implementing the access levels and authorization method disclosed herein includes levels of functionality that varies between the different types of users. Generally, the patient component includes basic functionality that is common to both components, and the physician component includes a number of physician-specific functions that are not pertinent or accessible to the patient. The differential functionality is important for maintaining the security and integrity of the patient's medical record account and the medical data network.

The patient component and/or the physician component may be provided, for example, in the form of a freestanding, installed software application or as a web-based program accessed via the Internet. Although the method is contemplated to be used over the Internet, it may alternatively be used over other networks, such as a private wide area network, or the like. With either component, a suitable network connection, for example an active Internet connection, is required. The user (e.g., patient or physician) is first presented with a logon screen that allows the user to log into the software in a conventional manner, for example via a user name and password system, and/or using a conventional peripheral device allowing for a biometric logon such as a fingerprint or retinal scanner, as are known in the art. Additional or alternative authentication systems may be utilized, such as key card or other token systems, or the like. The software application and controlling server software handles all data transmission protocols including interfacing with any audio and video device drivers of the host computer, collection and storage of all audio, video, and text data, encryption of all data (both command and informational data), transmission of the encrypted data to the intended recipient of the data stream and subsequent decryption of the data for viewing by the intended recipient. These functions are all typically transparent to the user.

Authorization Control

After logging in to either the patient component or the physician component, the user is presented an electronic medical record (“EMR”) access area. Within this area is a specific EMR authorization initiator, such as a software button, menu item or icon. Selecting EMR authorization opens a primary authorization window. On the patient component, the window consists of an “authorized physician” field, “outstanding authorization” field, and buttons to “add physician”, “remove physician”, “authorize”, “archive” or “close” the application. On the physician component, the window consists of an “authorized patient” field, “outstanding authorization” field, and buttons to “add patient”, “remove” patient, “authorize”, “archive”, “view document”, and “close” the application.

As indicated in the system diagram 100 of the FIGURE, control of the authorizations for the present method resides with the patient 102. The patient first establishes an account 104 for storing and accessing the patient's medical record and other health-related information. As discussed above, the patient has Level 1 account owner access, including full “read” access and limited “write” access. The patient may then elect to enter or otherwise upload personal information 106, for example demographic information, health and family history and/or symptoms and the like, into the patient's electronic medical record 150. Typical medical history information may include, for example, checklists that the patient may access and modify to indicate symptoms, medical history, current and formed medications, and the like. As discussed above, a person with Level 1-G guardian access privileges may also similarly access the patient's medical record account.

The patient will generally then designate one or more authorized users 108 of the patient's medical record account. For example the patient may authorized a Level 1-G guardian user, a Level 2 Physician Users 110, a Level 3 Institutional Users 112, and/or a Level 4 Ancillary Users 114. The designation of institutional users 112 may also enable emergency designation of Level 3-E access privileges, which designation may be completed solely through biometric means or the like, as discussed above, if the patient has turns this functionality on.

It is also contemplated that any Level 2 Physician User 110, or Level 3 (or Level 3-E) Institutional User 112 may provide limited read-only Level 4 Ancillary Services access 116 to particular records that the designating user has access to, for example to receive consultation, comply with court orders, or the like.

The Level 2 Author Access, and the Level 3 Institutional Access may be discontinued at any time by the Level 1 Owner of the medical record information. However, if the Level 2 Access or Level 3 Access of a healthcare provider is terminated by a patient, that healthcare provider retains read access to any medical record documents authored by that healthcare provider.

Therefore, when a Level 2 Access or Level 3 Access user attempts to access a patient's medical record, the system verifies that the user has current active privileges 120 to access the medical record. If the user has current active access 122, then that user can write 124 and read 126 any medical record documents in the patient's medical record 150. However, if the user does not have current active access 128, then the system checks to see if that user has ever created a medical record 130 in the patient's medical record account 150. If the user has never created a medical record 132 in the patient's medical record account 150, then access is denied 134. If the user has created a medical record 136 in the user's medical record account 150, then the system checks to see if the requested record is within the specified document range and time period 140 for which the user had access. If the requested medical record is not within the range accessible by the user 142 then access is denied 134, otherwise 144 the user is granted read-only access 126 to the requested medical record document.

Patient Control

The authorization process begins with the patient. In a current embodiment of the invention, the EMR authorization is activated using an authorization access, such as an icon, button or drop down menu, which opens the primary authorization window. An “add physician” button is selected to open a second window linked to a searchable database of all registered physician users. Registered physician users are listed alphabetically, and can be sub-classified or sorted by geographical location, specialty, and other searchable parameters. The desired physician may then be selected by either double-clicking on the identified entry, or highlighting it, and clicking an “Insert” button. This transfers the identified physician into a sub-field within the window. Clicking an “Update” button performs the simultaneous functions of placing an identifier for the identified physician entry into an “Authorized Physician” field of the patient's primary authorization window and placing the patient's name and demographic/unique identifier(s) into A physician-side “Authorized patient” window within the physician side primary authorization window. Preferably, the placement in the authorized physician or authorized patient window permanently associates the patient and authorized physician, although as discussed above the patient can terminate a physician's Level 2 access.

In front of each physician or patient name is an icon that denotes the status of that particular individual. In a preferred embodiment, a green icon is used to denote an active physician or patient within the list while a red icon is used to denote an inactive member of the list. In addition, there is a yellow indicator status that denotes a warning situation. While red, yellow, and green are the preferred color combinations, these are used for illustration purposes and any other color combinations could be used as well. This ensures that all patients have a traceable list of all physicians who had the ability to input data into their medical record while physicians have a traceable list of all patients whom they were able to view or input data into their medical record.

Physicians in the authorized physician window have full read/write Level 2 author access for this patient's medical record. Physicians are able to read records generated for this patient, and are authorized to create new medical record documents and store medical information within the patient's medical record. All transactions are permanently encoded with date, time, and author information as soon as the document is saved.

Once authorization has been granted, patients have the ability to revoke a particular physician's access to their medical record. Since physicians are the owners of the actual medical documents that they author, the physician retains the ability to view all of the medical documents that they created in this medical records account. Preferably, the physician also retains read access to the patient re-writeable core documents (demographics, past medical history, system review, etc) as created or modified during the time period where authorization was allowed. However, the physician will be denied access to all other aspects of the medical record, including any subsequent updates to the medical records account. In other words, the physician only retains access to the documentation as it was during the time the physician (or other Level 2 user) was authorized to access the medical record account and not reflective of the subsequent changes.

Deactivating a user's access is accomplished by selecting the physician to be deactivated from the authorized physician list, and clicking a “Remove” button. The remove function effectively identifies and preserves all of the physician-generated records and the core documents during the time of authorization, and assigns the physician Level 4 (read-only) account status for these documents. Although the physician's name remains within the “authorized physician” field, the status indicator changes from authorized (e.g., green-colored) to unauthorized (e.g., red-colored) to identify the physician as having limited read access to, and no write access to, the medical record. Within the physician's list of active patients, the icon associated with the patient also changes from authorized (green) to unauthorized (red) denoting the patient as inactive. “Unauthorized” (red icon) entries in either the patient or physician authorized user lists are maintained within the “authorized” window there for 30 days. If the patient wishes to restore access to the physician, selecting the entry and engaging or “clicking” the “Authorization” button will reinstitute their access and restore the appropriate icon to both the respective physician and associated patient lists. If record access remains denied for 30 days, the entry is automatically cleared into the archive. Clicking an “Archive” button brings up the archive window where these individual entries are stored.

Patients may also similarly grant records access to institutional and ancillary users. Selecting the appropriate institution from a list of institutional users located in the search field of the secondary authorization window will give the selected institution Level 3 or Level 4 privileges, depending upon what type of institution it is. Hospitals and institutions providing direct patient care are granted Level 3 privileges while ancillary users such as health insurers or lawyers are given Level 4 access only.

Physician Control

As individual patients grant their various physicians' authorization to access their medical record, this process automatically generates an active patient list for each practitioner. Patients authorizing a physician access to their medical record account have their name and a unique demographic identifiers listed in the physician's “Authorized Patient” field with each entry marked with the authorized (green) status icon. Physicians are able to select any patient from their authorized patient list and have complete Level 2 read/write privileges for the medical record account.

In some circumstances, such as outpatient follow-up after inpatient care, where a practitioner has been operating under institutional (Level 3) access, physicians may need to initiate a request to have access to an individual's medical record. In this instance, the physician would click on the “add patient” button. This opens a second window linked to a searchable database of all registered patient users. Unlike the physician search option where the universe of all physician users are listed alphabetically and a patient can browse the directory, the “patient search” database cannot be search in this fashion. The physician user can query the database by entering the patient's name, location, and other unique patient identifiers. When the proper entry is retrieved from the database, it is selected by either double-clicking on the entry, or highlighting it and clicking the “Insert” button. This function transfers the highlighted patient entry into a sub-field within the window. Clicking the “update” button performs the simultaneous functions of placing the physician's name into the “Outstanding Authorization” field of the patient's primary authorization window and the patient's name into the “Outstanding Authorization” field of the physician's primary authorization window.

If the patient wishes to grant the requesting physician access to their medical information, highlighting the entry in the “outstanding authorization” window and clicking the “authorize” button moves the both the patient and physician entry into their respective “authorized” windows, applies an active status indicators, and assigns Level 2 account access to the physician. If the patient does not wish to grant the physician account access, highlighting the entry and clicking remove or simply ignoring the request for a period of time, for example seven days, eliminates the entry from the “outstanding authorization” field. Placement in the authorized physician or authorized patient window is a permanent function. Once placed, those names are not removable. This ensures that all physicians have a traceable list of all patients for whom they have had the ability to create medical records. All transactions are permanently encoded with date, time, and author information as soon as the document is saved.

Physicians also have the ability to voluntarily discontinue a physician patient relationship. If a physician wishes to terminate such a professional relationship, they can select the appropriate patient from their active patient list and select remove. Unlike the process for a patient initiating the termination of a physician, when the physician initiates the process, the function opens an independent window displaying a “remove patient” protocol. The “remove patient” window imports the specific patient name and demographic information, as well as the physician name and demographic information. The form is time and date stamped at the time of the termination request. In order to prevent patient abandonment, states typically require the physician give the patient adequate notice, typically thirty days advance written notice, of the intent to terminate and must continue to see the patient for related care during that thirty day period. As a courtesy, they may also provide the name of another physician in the patient's local area who cares for similar problems. The “remove patient” form included all of the above information and an optional field for the physician to suggest another qualified practitioner. Clicking the “remove patient” button sends the message to the individual patient. If the patient is currently logged in, the “remove patient” message is displayed immediately to the patient. If they are not logged in, the next time the patient logs onto their account, the message is displayed. Once the message is displayed on the patient's side, the status indicator of the patient (on the physician's side) and the physician (on the patient side) changes to yellow. After the warning indicator, there is a numerical day counter function that counts backward from thirty. The number displayed denotes the number of days remaining before the relationship is terminated. Each time the patient account is opened during the thirty day time period, the “terminate patient” message window is displayed with the number of days remaining before account termination. During the warning period, physicians have full, Level 2 access. At day thirty-one, the yellow icons change to red and physician access is changed to read-only Level 4 access as described above. Inactive (red icon) entries in either the patient or physician authorized user lists are maintained within the “authorized” window there for thirty days. If record access remains denied for thirty days, the entry is automatically cleared into the archive. Clicking the “archive” button brings up the archive window where these individual entries are stored.

Physicians may be required to submit records to consultant physicians, insurers, attorneys, or other parties during the course of normal business. As such, physicians can assign these entities Level 4 “read-only” privileges to view specific documents or a range of documents which they have created within an individual's medical record. To perform this function, the physician selects the appropriate patient from the “authorized patient” or archive window, and clicks on the “document viewer” icon. Selecting the document viewer opens a new window similar to the physician search field. The physician has access to the physician user and institutional databases and selects the appropriate information target. A searchable list of all documents created by this particular physician within the patient's account (both under Level 2 and Level 3 authorizations) are listed in a separate window. The patient's record can be filtered by dates of service and each entry or range of entries, displayed with an associated check box. Checking the boxes associated with the desired documents to be forward moves copies of these documents into the document reader. Clicking the send button, forwards an entry into the “authorized” box of the target physician or institution.

Certain aspects, advantages, options and features of the current embodiment of the disclosed medical records system and access protocol are listed below, for illustrative purposes. It will be appreciated that not all of these aspects need be present in any particular embodiment of the present invention.

i. An networked information access system that allows variable levels of access to personal information, such as medical record information, depending upon a user classification.

ii. An Internet based medical record information access system that allows variable levels of access to personal and/or medical information, wherein access to the information is initiated and controlled by the patient.

iii. An Internet based information access system that allows variable levels of access to medical information between the account owner and another user that is initiated and controlled by the patient, wherein the system generates a list of available users and a list of authorized users for the patient.

iv. An Internet based information access system that allows variable levels of access to medical information that is initiated by the patient and that allows the health care provider to have access to multiple independent medical record accounts.

v. An Internet based information access system that allows variable levels of access to personal or medical information between the account owner and another user, that is initiated by the owner of the sensitive information which allows the end user to have access to multiple independent owner accounts and creates a list of active accounts.

vi. An information access system that includes an indicator system to denote active and inactive users.

vii. An information access system that includes an indicator system to denote active and inactive users.

viii. An Internet based information access system that allows variable levels of access to personal and or medical information that is initiated by the patient or owner of the sensitive information that allows the end user to terminate its access.

ix. An Internet based information access system that allows variable levels of access to personal and or medical information that is initiated by the patient or owner of the sensitive information that allows certain users the ability to change or add to the content of the personal sensitive information.

x. An Internet based information access system that allows the original user to limit, restrict, or revoke information access to a user once it has been given.

xi. An Internet based information access system that allows the author of any information or document added to the personal information record or medical record, to view the information they specifically added during the time period record access was granted irrespective of whether or not they currently have access to the sensitive information or record.

xii. An Internet based information access system that allows variable levels of access to personal and or medical information that is initiated by the patient or owner of the sensitive information and linked to a searchable database of potential authorized users.

xiii. An Internet based information access system wherein the database includes a list of physicians and/or health care providers.

xiv. An information access system wherein the database includes a list of healthcare institutions xv. An information access system wherein the database includes a list of insurers xvi. An information access system wherein the database includes a list of available ancillary users.

xvii. An Internet based information access system that has an archiving function that stores inactive users with previous account access.

xviii. An Internet based information access system that allows selection of a specific end user, and the ability of that end user to view a unique bit of information, document, or subset or information, in a time dependent fashion.

xix. An Internet based information access system which allows the original user to limit, restrict, or revoke information access to a user once it has been given, and allows certain users the ability to change or add to the content of the personal sensitive information

xx. An information access system that allows the potential end-user the ability to request account access from the account owner.

xxi. An information access system that will give access to multiple users within an institutional setting from a single authorization.

xxii. An information access system that will allow emergency read only access via a biometric log-on function.

xxiii. An information access system that will allow full read/write account privilege if the user is a registered institution.

xxiv. An information access system that will allow access for a legal guardian.

While illustrative embodiments have been illustrated and described, it will be appreciated that various changes can be made therein without departing from the spirit and scope of the invention. While illustrative embodiments have been illustrated and described, it will be appreciated that various changes can be made therein without departing from the spirit and scope of the invention.

The embodiments of the invention in which an exclusive property or privilege is claimed are defined as follows:

1. A method for controlling access to a patient's medical record information comprising the steps:
providing an electronic medical record account for storing a patient's medical records;
providing the patient with read access to the electronic medical record account over a computer network;
providing the patient with a list of registered healthcare providers that can be authorized to access the electronic medical record, such that the patient can select a registered healthcare provider;
identifying the selected registered healthcare provider as an authorized healthcare provider;
providing the authorized healthcare provider with read and write access to the electronic medical record account; and
recording individual medical records entered by the authorized healthcare provider into the electronic medical record account.

2. The method of claim 1, further comprising allowing the patient to select a plurality of registered healthcare providers and identifying the plurality of healthcare providers as authorized healthcare providers.

3. The method of claim 2, further comprising providing the patient with a list of authorized healthcare providers, such that the patient can select an authorized healthcare provider for deletion, identifying the selected authorized healthcare provider as a deleted healthcare provider, and withdrawing write access to the electronic medical record account for the deleted healthcare provider.

4. The method of claim 1, further comprising the steps of providing the authorized healthcare provider with a notification that the authorized healthcare provider has been identified as an authorized healthcare provider, and providing the authorized healthcare provider with demographic information about the patient.

5. The method of claim 4 further comprising providing the authorized healthcare provider with means for terminating the authorized healthcare provider's write access to the electronic medical record account.

6. The method of claim 1 wherein the electronic medical record account is accessible over the internet.

7. The method of claim 1, further comprising providing the patient with an option to allow emergency access to the electronic medical record account by healthcare institutions using a biometric identifier for the patient.

8. The method of claim 7, wherein the biometric identifier is selected from the patient's finger print and the patients retinal scan.

9. The method of claim 1, further comprising providing the patient with a list of healthcare facilities such that the patient can select a healthcare facility; identifying the selected healthcare facility as an authorized institutional user; and providing the authorized institutional user with read and write access to the electronic medical record account.

10. The method of claim 2, further comprising providing the patient and the authorized healthcare provider with a means for identifying documents in the electronic medical record account for read only access by a particular ancillary user; and providing the particular ancillary user with read only access to the identified documents for a limited period of time.

11. The method of claim 1, further comprising the step of providing the patient limited write access to the electronic medical record account such that the patient can enter personal demographic information into the electronic medical record account.

12. The method of claim 11, further comprising providing the patient with a medical history checklist, and wherein the patient limited write access further comprises access to enter data into the medical history checklist.

13. A method for controlling access to information in an electronic medical record account comprising the steps:
providing an account owner with a first software component for opening a patient electronic medical record account, the software component having fields for entering demographic information such that the account owner has full read access and limited write access to the electronic medical record;
registering a plurality of physicians that may be authorized to access the electronic medical record account, and providing the registered physicians with a second software component that is capable of accessing the electronic medical record account;
displaying with the first software component a list of the registered physicians such that the account owner can select one or more of the registered physicians;
providing the selected registered physicians with read and write access to the electronic medical record account through the second software component, such that the selected registered physicians can enter medical records into the electronic medical record account;
notifying the selected registered physician through the second software component that the registered physician has access to the electronic medical record account.

14. The method of claim 13, further comprising providing the selected registered physicians with means for terminating the selected registered physician's write access to the electronic medical record account.

15. The method of claim 13 wherein the electronic medical record account is accessible over the internet.

16. The method of claim 13, further comprising providing the account owner with an option to allow emergency access to the electronic medical record account by healthcare institutions using a biometric identifier for the patient.

17. The method of claim 16, wherein the biometric identifier is selected from the account owner's finger print and the account owner's retinal scan.

18. The method of claim 13, further comprising providing the account owner with a list of healthcare facilities such that the account owner can select a healthcare facility; identifying the selected healthcare facility as an authorized institutional user; and providing the authorized institutional user with read and write access to the electronic medical record account.

19. The method of claim 13, further comprising providing the account owner and the selected registered physicians with a means for identifying documents in the electronic medical record account for read only access by a particular ancillary user; and providing the particular ancillary user with read only access to the identified documents for a limited period of time.

20. The method of claim 13, further comprising providing a legal guardian of the account owner with read access to the electronic medical record account through the first software component.
